(19) United States
(12) Patent Application Publication
(10) Pub. No.: US 2018/0280606 A1
(43) Pub. Date: Oct. 4, 2018

(54) Nasal Flushing Catheter

(71) Applicant: Patent Wide International Corporation

(72) Inventors: Lih-Chiu Wu, Kaohsiung City (TW); Hui-Shuan Lee, Kaohsiung City (TW); Tsang-Mu Lee, Kaohsiung City (TW); Tze-Yu Lee, Kaohsiung City (TW)

(21) Appl. No.: 15/765,937

(22) PCT Filed: Oct. 14, 2015
(86) PCT No.: PCT/CN2015/091911

§ 371 Date: Apr. 4, 2018

(51) Int. Cl. A61M 3/02; A61M 25/00; A61L 29/06

(52) U.S. Cl. CPC A61M 3/0279; A61M 25/007; A61M 3/022; A61M 25/0015; A61M 2210/0618; A61M 2205/0216; A61M 2205/025; A61L 29/06

(57) ABSTRACT

A nasal flushing catheter, made of silicone latex, thermoplastic elastomer or other soft and elastic material, can be operated by the patients. The catheter has a closed end and an open end on the opposite side. There are multiple side-holes near the closed end. This catheter, when adapted to a connector and further connected to a syringe, can eject multiple strong thin spouts within the nasal cavity and nasopharynx for cleansing mucus and crust. The catheter may contain at least one stylet to improve its controllability. By a given injection rate of 10 cc/sec using a syringe, the average vertical height of spouts can be predicted by the total area of side-holes, the catheter is therefore classified as following: 1. a low pressure catheter, having 3.367 mm²˜4.123 mm² of total areas of side-holes to eject 30 to 45 cm spouts; 2. a medium pressure catheter, having 2.381 mm²˜3.367 mm² of total area of side-holes to eject 45 to 90 cm spouts and 3. a high pressure catheter, having less than 2.381 mm² of total area of side-holes to eject longer than 90 cm spouts.

### FIELD OF THE INVENTION

The present invention relates to a nasal flushing catheter, and more particularly to an innovative nasal flushing catheter for cleaning the mucus and crust deeply within the nasal cavity and nasopharynx to improve the symptoms of chronic sinusitis.

### BACKGROUND

Despite not life-threatening, the chronic sinusitis by the diagnostic criteria of symptoms lasting more than 12 weeks is a troubling disease. Currently, there is no cure for the disease. The main symptoms of nasal obstruction, foreign body sensation in the throat and cough would bother the patient relentlessly and may even lasting life-long. Antibiotics are only used for control of acute infection, and surgical operation is a method to remove the obstruction or to restore the patency of sinus orifice to prevent acute episode of sinusitis. No matter what the treatment is taken, the disease remains unchanged, and the symptoms persist. Hence, the otolaryngologists recommend long-term home-remedy of nasal flushing treatment at least twice a day.

Please refer to FIG. 1 and FIG. 2. FIG. 1 and FIG. 2 are respectively the sagittal sectional view and the coronal cross-sectional view of the human nasal cavity. The coronal section view of the nasal cavity shows a triangle configuration. The nasal cavity is divided into two spaces by the nasal septum. In each space, the nasal cavity proper located between the hollow nasal vestibule 90 anteriorly and the nasopharynx 91 posteriorly, is divided into three slightly curved slit-like passages as well as a para-septal space by three pieces of curved plate-like structure. The said three curved plate-like structures are called the superior turbinate A, the middle turbinate B, as well as the inferior turbinate C, and the said three curved slit-like passages are called the superior nasal meatus A1, the middle nasal meatus B1 and the inferior nasal meatus C1. The para-septal space can be divided into the superior para-septal space D1, the middle para-septal space D2 and the inferior para-septal space D3 by the lower edge of the superior turbinate A, the middle turbinate B, as well as the lower turbinate C. All the three turbinates, the nasal septum and the side wall of nasal cavity are constituted by bones and covered by mucosa to increase the surface area to ensure two functions: 1. Adjusting the humidity and temperature of inspired air. 2. Filtering the particulate substance in the air to avoid falling into the trachea and lungs (please see nasal cavity by Wikipedia). These three nasal passages, despite communicate with the para-septal space medially, the nasal vestibule 90 anteriorly and the nasopharynx 91 posteriorly, are independent to each other. All the mucus secreted from four pairs of sinuses located on both sides and superior aspect of nasal cavity, are discharged from the sinus orifices located at the roofs of the upper nasal meatus A1, the middle nasal meatus B1 and the nasopharynx. When the diagnosis of chronic sinusitis is confirmed, most patients already have severe symptoms and cannot recover to the normal conditions. Hence, it is difficult to recall how the disease occurs; however, its pathophysiology can be illuminated by the natural course of common cold.

The nasal secretions can be understood through a cold process. Running nose occurs in the early stage. The nasal discharge at this stage is a kind of clear liquid without mucus and can be completely evaporated. Afterwards, the discharge becomes tenacious and difficult to expel which is called mucus. Then, the secretion becomes jelly-like scab, and finally solid booger (crust). The mucus, scab and crust are the three states of mucus. The symptoms of chronic sinusitis are due to accumulation of these three patterns of mucus within the nasal cavity body. The symptoms of common cold rarely last more than 4 weeks, while the symptoms of chronic sinusitis persist much longer. Only chronic sinusitis has symptoms persisting more than 12 weeks, therefore, can be used as a diagnostic criteria. If no other obstructive causes such as polyps or tumors present, the diagnosis can be confirmed. The allergic rhinitis or hay fever so on may also present with severe nasal obstruction persisting from a long time, but is always accompanied by a lot of watery nasal discharge and absence of foreign body sensation in the throat. As long as the allergen disappears, the symptoms disappear. In addition, allergic rhinitis is a periodic and seasonal disease, while chronic sinusitis is a life-long persisting disease.

The so called nasal obstruction is a sense of exertion effect on respiration due to blockage of nasal passage. Because the nasal passage is composed of front to back slit like spaces, significant area reduction of these spaces at any coronal plane of nasal cavity may cause nasal obstruction. In the early period of chronic sinusitis, the mucus secreted into nasal cavity from sinuses, does not cause significant area reduction of nasal passage. Due to its stickiness, the slow migration of mucus by gravity precludes the appreciation of its presence. Hence, the patient would not consciously expel it out. The mucus migrates by gravity to form a thin layer onto the turbinate. Because the respiratory air is not block in this area, the mucus in thin layer is quickly dried by the respiratory air to become even much thinner crust coating on the turbinate. Repeated coating of the crust results in gradual enlargement of turbinates until the severe narrowing of nasal passage. Meanwhile due to the aerodynamics, the configuration of the turbinate is retained and may be difficult to distinguish from as that caused by mucosal swelling in allergic rhinitis. The situation progresses until significant nasal obstruction forces the patient to seek medical treatment. At this time, the nasal passages become narrow, even the viscosity of mucus remains unchanged, it is more difficult to expel due to increased relative viscosity, and finally it causes complete obstruction beginning from the upper part of nasal cavity. This is so called insidious onset of symptoms. The mucus continuously discharges from the opening of the upper nasal meatus and the middle nasal meatus, and spreads from the top to down. This causes a suspended conglomerated mass on the upper part of nasal cavity while the lower part of nasal cavity remains more patent. The downstream of mucus still contacts with the respiratory air, and becomes the hardest crust state (shown in FIG. 3). Even the mucus is difficult to expel, it still a liquid and could move with the gravity. If the mucus moves forward to nasal vestibule 90, it is the known as nasal mucus and could be further dried to booger. The mucus may also move backward to the nasopharynx 91, known as post-nasal drip, and it is difficult to clean by effort of suctioning or swallowing. The mucus in the nasopharynx has the same destiny as that in the nasal cavity, could become crust. Repeated coating results in thick crust and mucus on the walls of nasopharynx which is the source of copious mucus in the mouth of patient. Copious mucus in the mouth is a reliable diagnostic criteria for chronic sinusitis, but is not a great suffering to the patient and does not include in the three main symptoms. Both the liquid mucus and solid booger may fall downward and have the possibility to fall into the trachea to cause foreign body sensation in the throat and initiate a defense mechanism of cough. The foreign body sensation in the throat and cough is also a great suffering, and may even restrain the social activities of patient. The symptoms of nasal congestion, foreign body sensation in the throat and cough may also affect the sleeping quality resulting in poor quality of life and poor working performance. Finally, if these symptoms are ignored, the mucus as well as the crust will eventually block the orifice of the paranasal sinus. This will result in the accumulation of mucus in the paranasal sinus to cause acute sinusitis. Acute sinusitis is a serious disease required vigorous medical and/or surgical treatment. After the acute episode is controlled, the disease and the symptoms remain the same.

The interchange of mucus, jelly-like scab and solid booger (crust) can be observed in in-vitro experiments. The mucus exposed in the air will become jelly-like scab, and the jelly-like scab will become solid booger. The volume may shrink more than 10 times from liquid mucus to solid booger. The duration depends on the degree of humidity and the air circulation. Conversely, if the solid booger is soaked in the water for one hour, it will become half-solid scab, and then it gradually becomes mucus. This is the rational of nasal flushing treatment for chronic sinusitis and all medical experts recommend conventional nasal flushing device for this treatment. However, some of our family members and our friends with chronic sinusitis followed the instruction of medical expert using conventional nasal flushing device, the symptom improvement was very limit initially and gave up quickly due to unsatisfactory therapeutic effect.

All conventional nasal flushing devices are similar in basic structures. Please refer FIG. 4, Taiwan patent No. M418689 disclosed a nasal irrigation device having an applying portion 10 and a nozzle 20. The applying portion 10 has an inner space 11 and the nozzle 20 has an opening 21 and an extending tube 22 reaching towards to the inner space 11 of the applying portion 10. When the inner space 11 is filled with water and compressed to push the water into the extending tube 22 and exit from the opening 21.

The fundamental problem of conventional nasal flushing devices is its direction of spouts. These devices can only produce frontal back single directional spouts by a big nozzle placed in nasal vestibule. Although this single directional spouts can rinse the whole nasal cavity in normal person, this spouts can only pass through the wide and non-obstructed inferior meatus to nasopharynx but cannot reach the narrow and obstructed middle and superior meatus in severe cases of chronic sinusitis. These narrow and obstructed areas can only be rinse effectively by multi-directional spouts perpendicular to the nasal passage by a catheter placed in middle and superior nasal meatus. Therefore we filed a patent application as following.

Please refer to FIG. 5, Taiwan patent No. 101125880 disclosed a nasal flushing device having a catheter body 31 is comprised with a closed end and a plural of openings 32. The said catheter body 31 can be inserted into the middle para-septal space D2, middle nasal meatus B1, the inferior nasal meatus C1 and nasopharynx to eject multidirectional spouts perpendicular to the catheter body 31. These multidirectional spouts can reach 30 cm long. Before the patent application, manual-made nasal flushing catheter has been used among our family members and close friends with chronic sinusitis. The efficacy was much better than conventional nasal flushing devices, confirming the fundamental problem we found, but was not completely satisfactory in some of these patients. Although a catheter small enough to advance into superior nasal meatus was too soft to control and a catheter having a good controllability was too large to be inserted into the deep recess of nasal meatus, but the maximal height of nasal cavity is 5 cm while the length of spouts is 30 cm, why the 30 cm spouts cannot reach the 5 cm deep recess areas? We therefore conducted an in-vitro experiment to answer this question.

We first collected a lot of mucus to paste on many plastic plates and left the mucus dried to become solid crust. These plastic plates were kept in upright position to avoid fluid stasis for simulating the nasal meatus in flushing condition. The crust areas of the plastic plates were flushed by 3 different strength spouts and variable angles related to the up-right plastic plate. The strength of the spouts was classified as following: 1. Low pressure spouts under 45 cm vertical height; 2. Medium pressure spouts between 45 to 90 cm vertical height and 3. High pressure spouts over 90 cm vertical height. The relationship between strength as well as angle of spouts and the fluidity of crust after flushing were observed as following: 1. the stronger the spout, the better fluidity of crust after flushing, 2. vertical angle spout has the best flushing effect. The results of this experiment can explain the unsatisfactory effect of the prior nasal flushing catheter as following. Since the nasal cavity is a slit like air passage, when the catheter is too large and can only be placed at the inferior wide portion of middle nasal meatus or the para-septal space, only the spouts parallel and aligned to the slit can pass through the slit, but they are parallel spouts related to the slit, therefore the crust has to be penetrated is the depth of the slit measured in centimeter range which may require strong spouts to achieve the purpose; but if the catheter is small enough to be placed at the deep narrow recess of middle and superior nasal meatus, the crust at this target area can be rinsed directly by vertical spouts and the crust has to be penetrated is the thickness of crust measured in millimeter range, this only require weak spouts. Centimeter and millimeter is a huge difference around 10 times. The 30 cm spouts by the prior nasal flushing catheter placed at inferior wide portion of nasal meatus or middle para-septal space D2 is obviously inadequate to effectively rinse the deep narrow recesses of nasal meatus illuminated by the above experiment. Further, when the parallel spout entering the narrow slit space, the crust within this space can absorb the water to expand its volume causing more severe nasal obstruction; this explains why 30 cm spouts are not completely satisfactory in nasal flushing treatment.

These are the fundamental problems of our prior nasal flushing catheter. The resolutions are 1. To increase the strength of spouts without increasing the outer diameter of catheter, and 2. To make better controllability of small catheter.

On the priority date of the present patent application (Oct. 14, 2015), the prior nasal flushing catheter has not been patented and not commercialized. All medical experts in the world by then still recommended conventional nasal flushing device for nasal flushing treatment (chronic sinusitis-Medscape reference; chronic sinusitis-Mayo Clinic) and the Sinus Rinse of NeilMed pharmaceutical INC. was a favorite conventional nasal flushing device in the United States of America. Because the prior nasal flushing catheter was only a document among the vast if not indefinite data of patent applications in the whole world, no-body paid attention to this document and the advantage of the prior nasal flushing catheter could not be appreciated. Since the catheter was not commercialized and its advantage was not appreciated, it was impossible to try this catheter to find its disadvantage. Therefore, the fundamental problems of the prior nasal flushing catheter was impossible to be discovered by any other person, not mention to conceive these solutions, we therefore file this patent application.

### SUMMARY OF THE INVENTION

Therefore, the purpose of the present invention is to provide a better flushing catheter having stronger spouts and/or better controllability for entering the narrow crooked nasal passages.

The flushing catheter includes a catheter body and a connector formed outside of the catheter body. The said catheter body is made of silicone, latex, thermoplastic elastomer or other soft and elastic materials, therefore can be operated by patient to place into the nasal cavity and nasopharynx. The catheter body has a closed end, an open end on the opposite side. Multiple side-holes are made near the closed end of the catheter body.

Another technique of an embodiment of the present invention is that the catheter body is not larger than 1.0 mm in outer diameter.

Another technique of an embodiment of the present invention is that the catheter body is not larger than 1.0 mm in outer diameter. The side-holes are linear in shape and parallel to the catheter body.

Another technique of an embodiment of the present invention is that the flushing catheter further includes a tapered end metallic stylet undergone anticorrosive treatment providing within the lumen of the nasal flushing catheter.

Another technique of an embodiment of the present invention is that the flushing catheter further includes multiple different length and uniform diameter metallic stylets undergone anticorrosive treatment providing within the lumen of the nasal flushing catheter.

Another technique of an embodiment of the present invention is that the stylets are made of tungsten alloy undergone anticorrosive treatment with diameter not larger than 0.1 mm.

Another technique of an embodiment of the present invention is that the total area of multiple side-holes on the catheter body is between 3.367 mm²˜4.123 mm² in range.

Another technique of an embodiment of the present invention is that the total area of multiple side-holes on the catheter body is between 2.381 mm²˜3.367 mm² in range.

Another technique of an embodiment of the present invention is that the total area of multiple side-holes on the catheter body is smaller than 2.381 mm².

Another technique of an embodiment of the present invention is that the plurality of side-holes is more densely distributed when closer to the closed end.

The advantages of this invention are to improve the efficiency and efficacy of nasal flushing treatment by optimal strength of spouts and to improve the controllability of the small and soft catheter by the stylet inside the catheter, and by the design of the connector and total area of side-holes, the stronger thin spouts can be produced.

For the examiners to clearly understand the technical features and the content of the present invention, the present invention is hereinafter described in details with the accompanying drawings and the expression in the form of two preferred embodiments. In addition, prior to the detailed description, it should be noted that same or similar reference numerals are used to designate the same or similar elements.

### BRIEF DESCRIPTION OF THE DRAWINGS

FIG. 1 is a sagittal section view of the human nasal cavity.

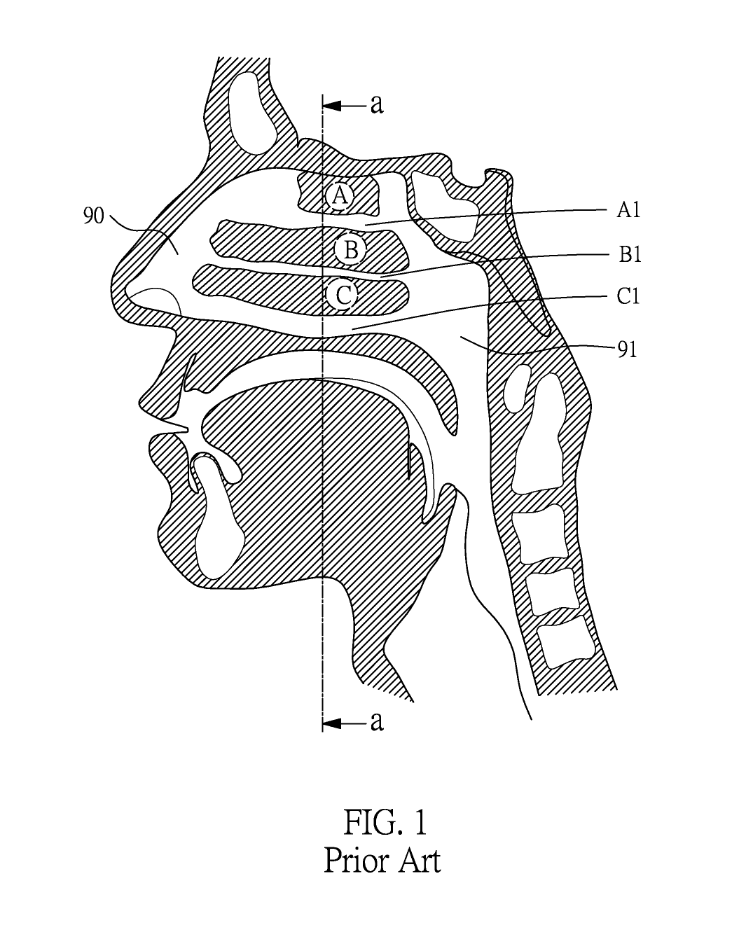

FIG. 2 is a coronal section view of middle portion of nasal cavity along the a-a line in FIG. 1.

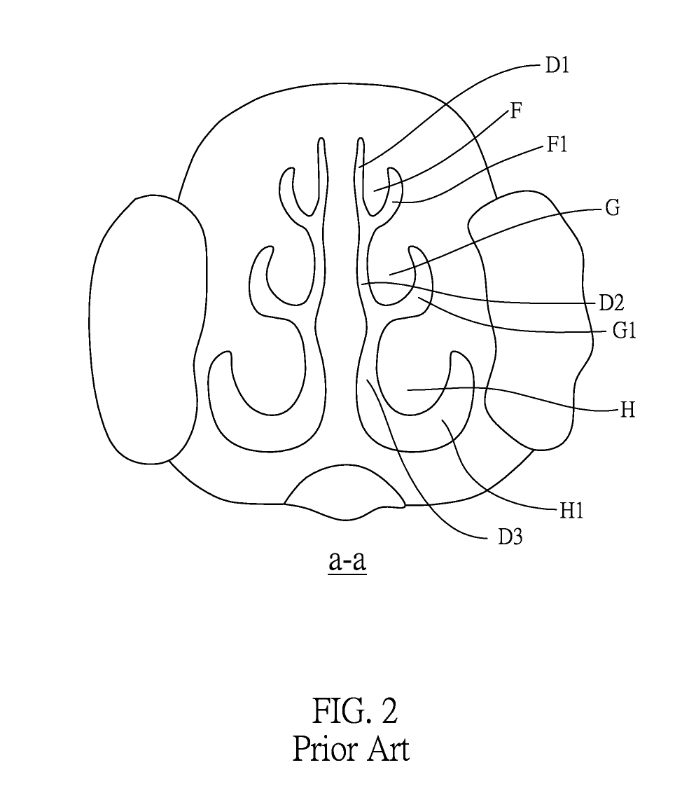

FIG. 3 is a coronal section view of the middle part of the nasal cavity showing the extension of mucus and crust from superior para-septal space, superior nasal meatus and middle nasal meatus to inferior para-septal space in a patient of chronic sinusitis.

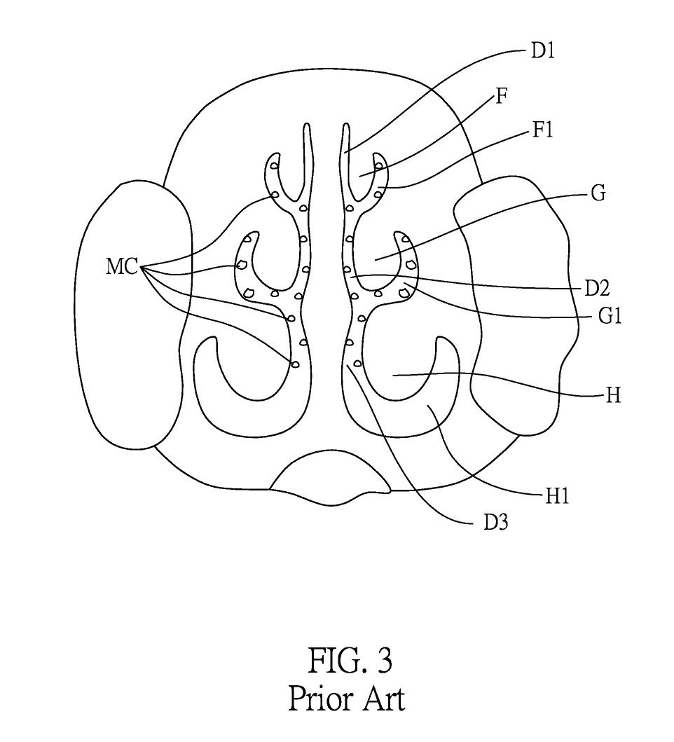

FIG. 4 is a cross-sectional view of a nasal irrigation device disclosed in Taiwan patent no. M418689.

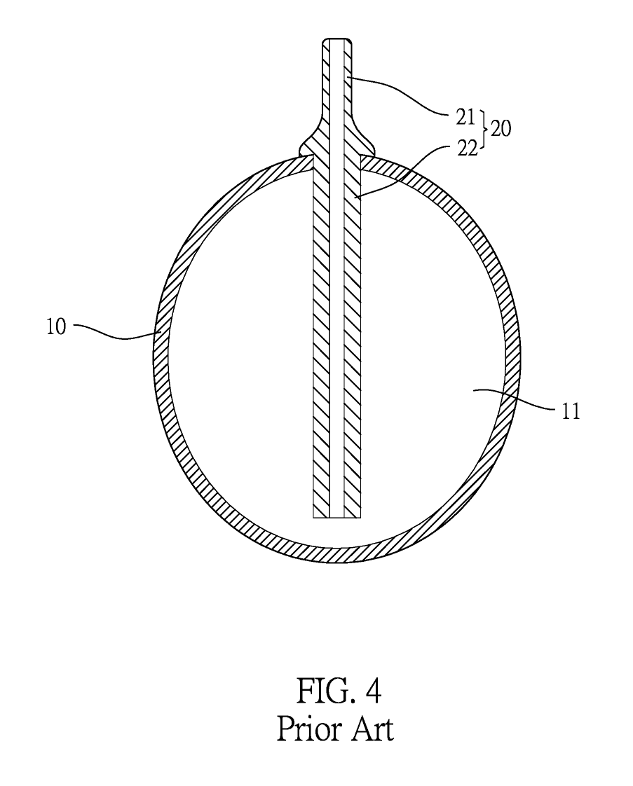

FIG. 5 is a perspective view of a nasal flushing catheter device disclosed in Taiwan patent No. 101125880.

FIG. 6 is a perspective view of the nasal flushing catheter according to the first embodiment of the present invention.

FIG. 7 is a partial top view of the nasal flushing catheter showing denser distribution of the plurality of side-holes closer to the closed end.

FIG. 8 is a partial cross-sectional view of the nasal flushing catheter according to the second embodiment of the present invention.

FIG. 9 is a partial cross-sectional view of the nasal flushing catheter according to a third embodiment of the present invention.

FIG. 10 is a partial cross-sectional view of the nasal flushing catheter according to a fourth embodiment of the present invention.

FIG. 11 is a partial cross-sectional view of the nasal flushing catheter according to a fifth embodiment of the present invention.

FIG. 12 is a partial cross-sectional view of the present invention, showing the connection condition of a connector and the catheter body.

FIG. 13 is a partial cross-sectional view of the present invention, showing the connection condition of a connector and the catheter body as well as the safety design of the stylet.

FIG. 14 is a coronal section view of the middle part of the nasal cavity, showing the crooked and narrow paths of the superior nasal meatus and the middle nasal meatus.

FIG. 15 is a sagittal section view of the human nasal cavity, showing the catheter placed in the middle nasal meatus for flushing.

FIG. 16 is a sagittal section view of the human nasal cavity, showing the catheter placed in the upper nasal meatus for flushing.

FIG. 17 is a coronal section view of the human nasal cavity, showing the catheter placed in the inferior para-septal space for flushing.

FIG. 18 is sagittal section view of the human nasal cavity, showing the catheter placed in the nasopharynx for flushing.

### DETAILED DESCRIPTION OF THE PREFERRED EMBODIMENTS

The relevant features and the technical fields of the present invention will be illuminated by the description of the preferred embodiments as well as the accompanying drawings.

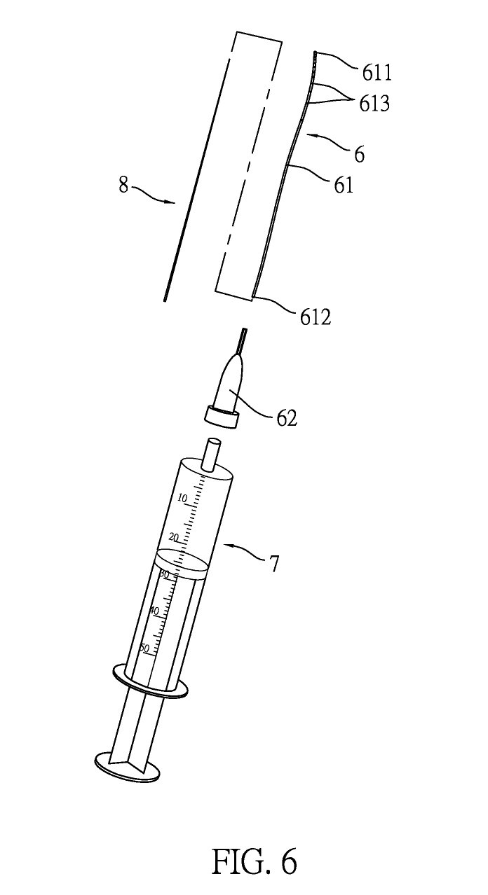

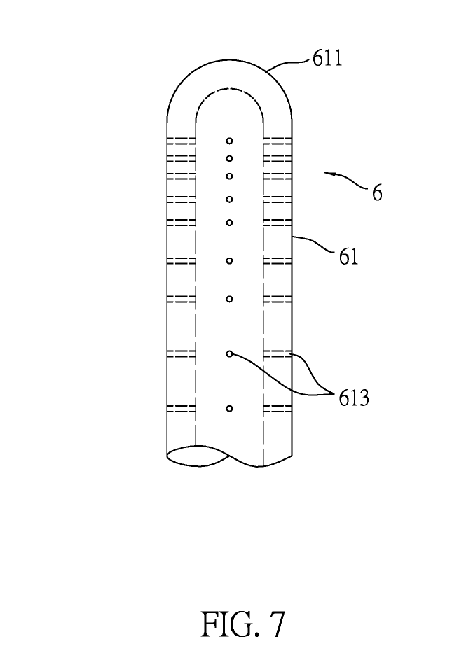

Please refer to FIG. 6 and FIG. 7, showing the nasal flushing catheter 6 of the first preferred embodiment of the present invention. In the first preferred embodiment, the nasal flushing catheter 6 can be adapted to a syringe 7 for rinsing the nasal cavity and nasopharynx of the human body.

In the first preferred embodiment, the nasal flushing catheter 6 includes a catheter body 61, and a connector 62 formed outside of the catheter body 61. The said catheter body 61 is made of silicone, latex, thermoplastic elastomers or other soft and elastic material which can be operated by patient to put into the nasal cavity and nasopharynx. The catheter body 61 has a closed end 611 and an open end 612 on the opposite side of the closed end 611. A plurality of side-holes 613 is circumferentially distributed in a segment near the closed called flushing section 610. The plurality of the said plurality of side-holes 613 may be evenly distributed or denser distribution closer to the closed end 611 (as shown in FIG. 7).

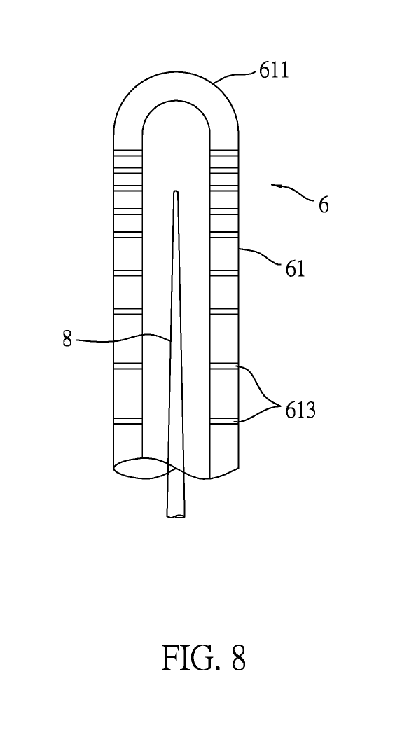

Please refer to FIG. 8. FIG. 8 shows the nasal flushing catheter 6 of the second preferred embodiment of the present invention. This second preferred embodiment is substantially similar with the first preferred embodiment. The similarities are omitted here, and the difference is presence of a metallic stylet 8 within the catheter. This stylet has been undergone anticorrosive treatment and has a tapered end.

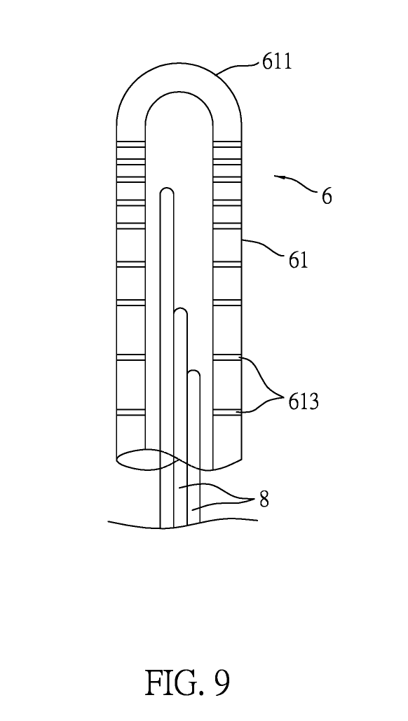

Please refer to FIG. 9. FIG. 9 shows the nasal flushing catheter 6 of the third preferred embodiment of the present invention. This third preferred embodiment is substantially similar with the second preferred embodiment. The difference is that the metallic stylet 8, here the stylets are uniform in diameter but different in length. The number of metallic stylet 8 in this embodiment is 3. In the actual implementation, it is not restricted in three stylets and could be other numbers of stylets.

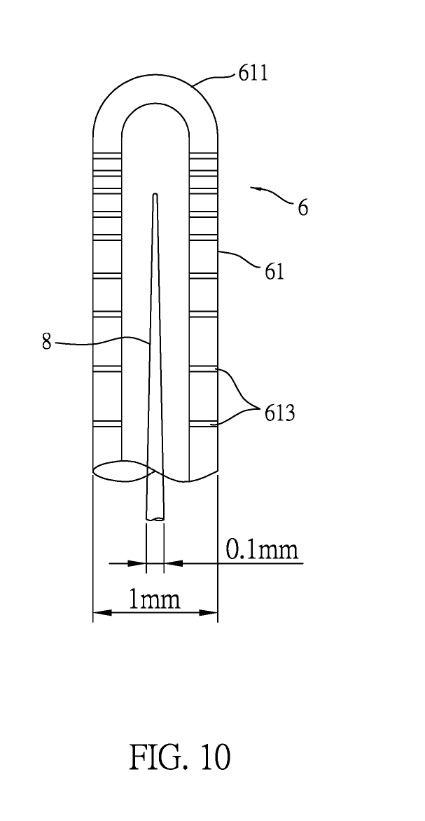

Please refer to FIG. 10. FIG. 10 shows the nasal flushing catheter 6 of the fourth preferred embodiment of the present invention. The fourth preferred embodiment is substantially similar to the second or third preferred embodiment except some differences. The differences are the outer diameter of the catheter body 61 and the material of stylet. The outer diameter of the catheter body 61 is not more than 1.0 mm, and defined as the micro-catheter. The stylets are made of tungsten alloy undergone anticorrosive treatment with diameter not larger than 0.1 mm.

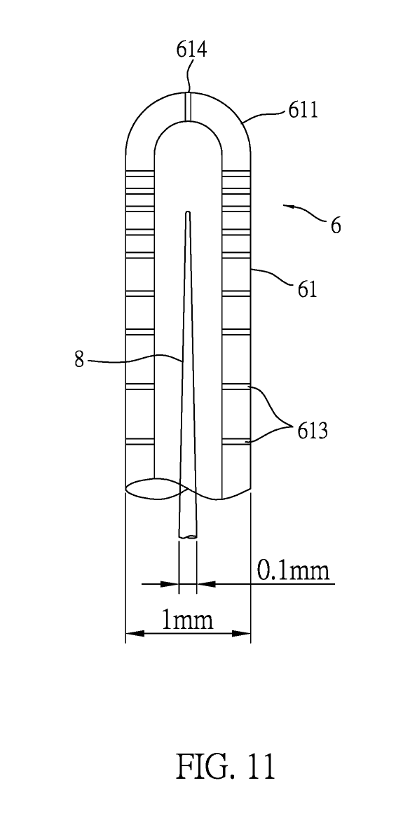

Please refer to FIG. 11. FIG. 11 shows the nasal flushing catheter 6 of the fifth preferred embodiment of the present invention, which the fifth preferred embodiment is substantially similar with the fourth preferred embodiment except an additional small end-hole 614 is formed at the closed end side 611 of the catheter body 61. The nasal flushing catheter 6 further includes a stylet 8 made of tungsten alloy undergone anticorrosive treatment with diameter not larger than 0.1 mm and tapered end.

The following is the detailed description about the manufacture of catheter, the relationship between the side-holes and the height of the vertical spout, the manufacture of side-holes as well as their arrangement, the relationship between the catheter and the side-holes, the design of stylet, the connector, the selection of the syringe, the actual operation, and the improvement of the therapeutic effect for nasal flushing catheter 6 of the present invention.

The Manufacture of Catheter

The said catheter body 61 is made of silicone, latex, thermoplastic elastomer or other soft and elastic materials. The said catheter body 61 has a closed end 611, an open end 612 on the opposite side of the closed end 611, and a flushing section 610 near the closed end having a plurality of side-holes 613 disposed circumferentially. The widest inferior nasal meatus H1 and the inferior para-septal space 13 of the nasal cavity, are not wider than 4 mm in normal person, and may be 2.5 mm or even narrower than 1.5 mm in patients with chronic sinusitis. The narrowest superior nasal meatus, is usually not wider than 1.5 mm in normal person, and even narrower than 1 mm in patients with chronic sinusitis. Therefore, the outer diameter of the catheter of the present invention is not larger than 4 mm, and preferably, between 0.5˜2.5 mm. A catheter with outer diameter less than 1.0 mm is called a micro-catheter. A micro-catheter may have 0.6 mm or even 0.3 mm inner diameter. Because micro-catheter may be easy to burst during flushing action, an additional small end-hole may reduce the possibility of such risk. The wall thickness of catheter is less than 1 mm, and preferably between 0.1˜0.6 mm.

The Relationship Between the Side-Holes and the Height of the Vertical Spout

The average initial velocity (V₀) of side-holes 613 spout can determine the average vertical height h. The equations can be deducted as followings: V₀=_{g}t or t=V₀/g, wherein, g is the gravitational constant 9.8 m/sec², t is the time reaching the vertical apex. The maximum vertical apex h, can be determined by the equation h=1/2(gt²), plugging t=V₀/g, and h=1/2(g)(V₀/g)²=V₀²/2_{g} or V₀²=2gh; furthermore, the average initial velocity (V₀) of side-holes 613 spout can be determined by the injection rate (IR) and the total area of side-holes (TA), the equation is as following: V₀=IR/TA, when applies to the function V₀²=2gh, and then will get (IR/TA)²=2gh, or TA²=IR²/2gh, or TA=\sqrt{IR²/2gh}. Even the 10 cc, 20 cc, 50 cc, 60 cc, and 100 cc syringes 7 are all available easily, for considering the operability and avoiding choking phenomenon by large flow of water, the 10 cc syringe 7 may be the best choice. Hence, the 10 cc syringe 7 was chosen as a standard syringe of the present invention. Manual injection of a 10 cc syringe may reach a injection rate (IR) from 5 cc/sec to 15 cc/sec, hence, the medium value of 10 cc/sec is chosen as the standard injection rate. Under this standard injection rate, spout of 30 cm can be expected by side-holes 613 with total area TA=\sqrt{100/(2×9.8 m×0.3 m)}=4.123 mm²; similarly, spout of 45 cm can be expected by side-holes 613 with total area TA=\sqrt{100/(2×9.8 m×0.45 m)}=3.367 mm² and spout of 90 cm can be expected by side-holes 613 with total area TA=\sqrt{100/(2×9.8 m×0.9 m)}=2.381 mm². In conclusion, a catheter with a total area of side-holes 613 between 3.367 mm²˜4.123 mm² to produce 30˜45 cm low pressure spouts is called a low pressure catheter; a catheter with a total area of side-holes 613 between 2.381 mm²˜3.367 mm² to produce 45˜90 cm medium pressure spouts is called a medium pressure catheter; a catheter with less than 2.381 mm² total area side-holes 613 to produce over 90 cm high pressure spouts is called a high pressure catheter. Due to the prior invention can already eject 30 cm spouts, the low pressure spout is defined as 30˜45 cm.

The Manufacture and Arrangement of Side-Holes

The said side-holes 613 can be produced by traditional method of removing a part of the catheter wall, and can also be produced by puncturing method using a solid metallic needle with a conical tip. The size of side-holes is between 0.1 mm and 1 mm, and preferably between 0.2 mm and 0.5 mm. The number of 0.5 mm diameter side-holes can be made from 4.123 mm² total area is 4.123 mm²÷0.5 mm÷0.5 mm÷0.785=21 the number of 0.3 mm side-holes is 58 and the number of 0.2 mm side-holes is 131. The number of 0.5 mm diameter side-holes can be made from 3.367 mm² total area is 3.367 mm²÷0.5 mm÷0.5 mm÷0.785=17 the number of 0.3 mm side-holes is 47 and the number of 0.2 mm side-holes is 107. The number of 0.5 mm diameter side-holes can be made from 2.381 mm² total area is 12, the number of 0.3 mm side-holes is 33 and the number of 0.2 mm side-holes is 75.

During forceful injection, the closer to the closed end it is, the higher the intraluminal pressure is. Therefore, the side-holes 613 are designed to be more densely disposed near the closed end 611 (as shown in FIG. 8), or a shorter flushing section 610 is designed to avoid the over expansion at the distal end to endure higher pressure for ejecting the strongest spouts. Under a given total area of side-holes 613, the smaller the side-holes 613 are, the more side-holes 613 can be disposed, and the more they are widely distributed, which the wider area can be flushed. Under a given quantity of the side-holes 613, the smaller the side-holes 613 is, the smaller the total area is so that the stronger the injecting spouts are produced. Besides, the micro-catheter can only endure very little pressure, and can be used in a very narrow space without high pressure spout. Its purpose is for evenly transporting flushing liquid in the narrow slot pace, and using blade or laser to produce linear side-holes parallel to the catheter, so that the pressure in the catheter can be lowered during operation without affecting the catcher structure and the total area of side-holes can be increased.

The Relationship Between the Catheter and the Side-Holes

The height of spout can be determined by the injection rate (IR) and the total area of side-holes (TA), but only when the catheter can endure high pressure. Because of nasal cavity size, the catheter cannot expand unlimitedly, and the height of spout cannot either. Therefore, the height of high pressure spouts is defined as above 90 cm. Under a given outer diameter of the catheter body 61, the thinner the wall of the catheter is, the larger the inner diameter is and the lower the resistance on forceful injection, but easier to cause elastic fatigue. The said elastic fatigue will cause dilatation of the side-holes, increasing the total area of side-holes 613 and reducing the strength of spouts. However, the thicker wall and the smaller inner diameter of the catheter may cause smaller flow rate and greater resistance, which is not good for forceful injection to eject strong spouts. Therefore, the wall thickness and the inner diameter must be balanced. Although the stronger spouts may be more effective, however, stronger spouts may cause tingling sensation, and is also accompanied with smaller total area resulting in smaller number of side-holes that only smaller area can be cover during the flushing action, in other words, it is less efficient. Therefore if the symptoms are not so bad, the mucus is not so sticky and the solid crust is not so much, then the medium pressure or low pressure spouts may be enough to improve the symptoms. Besides, the area of side-holes for medium pressure or low pressure catheter is larger, the resulting larger number of side-holes can be disposed longer on the catheter to cover a larger area during flushing action, in other word, medium pressure or low pressure catheter is less effective but more efficient. Therefore, the three kinds of catheters have their definite indication, and no one shall be given up.

The Design of Stylet

A catheter with larger outer diameter and thicker wall has better controllability. However, a catheter should be smaller than the narrowest part of the insertion route to the target area of nasal cavity, a catheter smaller than 2 mm or even 1.0 mm in outer diameter may be difficult to operation and advancement. In addition to the hardness of the catheter, a gradual transition from hard to soft toward the distal close-end is necessary for excellent controllability. An easy solution to this problem is to put metallic stylet within the lumen of catheter. The metallic stylet should be undergone anticorrosive treatment. The metallic stylet 8 may be single in number having a tapered end or multiple in number with uniform diameter and different in length (as shown in FIG. 8 and FIG. 9). Furthermore, in some patients, the narrowest place of the route reaching the upper nasal meatus, the middle nasal meatus or superior para-septal space might be less than 1.0 mm, and even might be only 0.5 mm which can only be overcome by even smaller catheters. The inner diameter of such micro-catheter might be small as 0.6 mm or even 0.3 mm. Therefore, the stylet 8 must be further smaller, and the tungsten alloy is the choice for such stylets 8 which meets the requirement of smallest diameter and largest hardness, because the tungsten is the hardest metal (as shown in FIG. 10 and FIG. 11).

The Connector

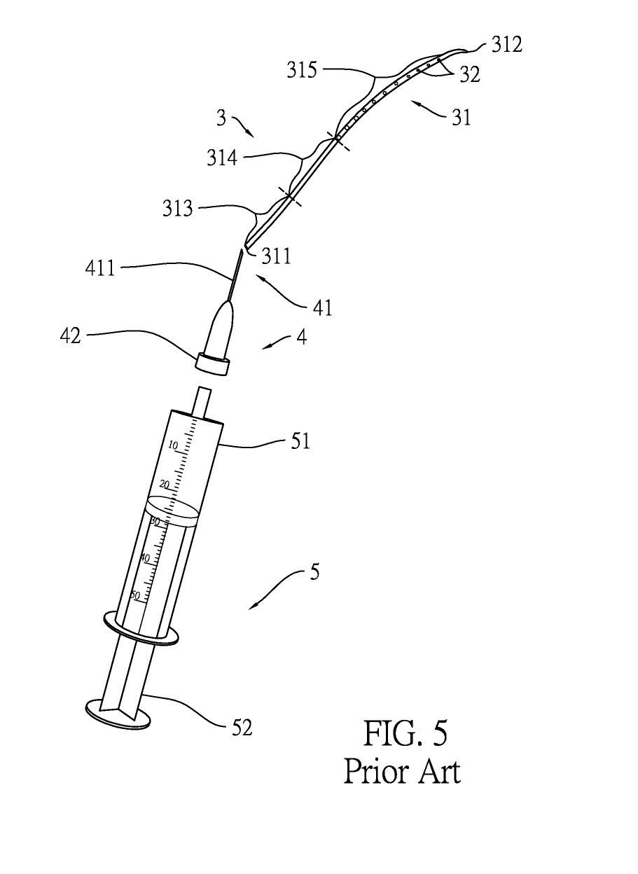

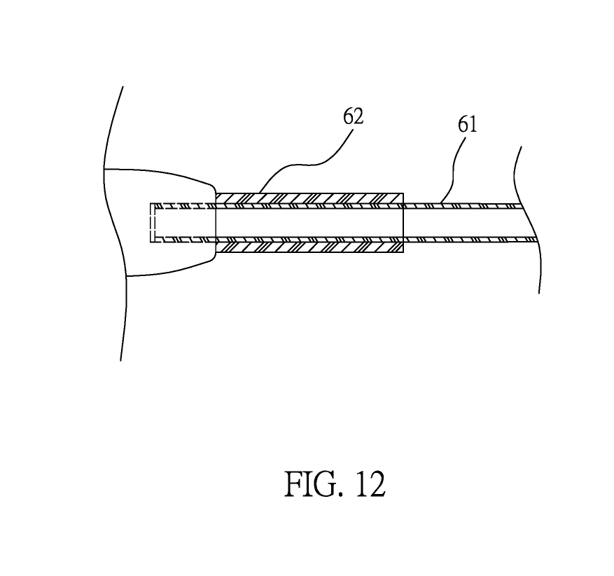

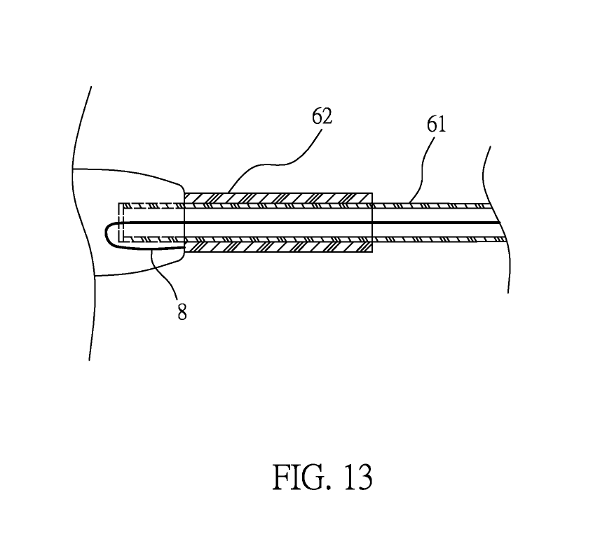

Please see FIG. 5, the Taiwan patent No. 101125880 disclosed a nasal flushing device, the outlet of the connector is inserted into the inlet of the catheter body. Not only the connector restricts the water flow, but also the connecting part of the catheter body is expanded to have thinner wall and hence cannot endure the pressure of injection. Therefore, the outlet of the connector 62 of this present invention is wrapped in the inlet of catheter by bigger inner diameter to solve the above mentioned problems. The security of connecting place can be accomplished by friction, powerful adhesive agent, thermoplastic change or other means. Furthermore, due to the very small inner diameter of the micro-catheter and the inside disposition of the stylets 8, the wrapping connector 62 makes more space to accommodate the stylets (as shown in FIG. 12). The stylets 8 can have a safe design at the connecting place to avoid forward moving during flushing action (as shown in FIG. 13).

The Selection of the Syringe

Even the 20 cc, 30 cc, and 60 cc syringes 7 are all available, they are not easy to operate due to their large size; therefore, a 10 cc syringe is the choice. However, the syringes 7 must be adjusted according the diameter of the catheter body 61. A catheter body 61 over 2 mm in outer diameter may use 10 cc syringe 7; a catheter body 61 with 1.5˜2 mm outer diameter may use 5˜10 cc syringe 7; a catheter body 61 having 1˜1.5 mm outer diameter may use 1˜5 cc syringe 7; A micro-catheter with less than 1 mm in outer diameter may use 1˜2 cc syringe 7 in order to avoid bursting of the catheter.

The Actual Operation

The handmade catheter of the prior nasal flushing catheter had been tried by our family members and close friends before filing the present invention for more than 2 years, meanwhile, the catheter of the present invention has also been tried for more than one year, hence, we have a lot of experiences and are familiar with all details of flushing treatment. Although the computed tomography can objectively assess the efficacy, the subjective assessment by the patient is more important. There are two issues for assessing the therapeutic effect: one is the amount of mucus wash out during or after flushing, the more the amount of mucus the better it is; the other is subjective feeling of symptom improvement, such as the severity of nasal obstruction, foreign body sensation in the throat and cough, of course these symptoms related to quality of sleep and daytime activity. Both of them have to be considered. If a patient using the catheter of the prior invention (FIG. 5) and inserts deeply enough within the nasal cavity (at least inserts by over 8˜10 cm), but still not satisfied with the flushing effect, the possible explanation may be that: the catheter was not inserted into the target areas of middle nasal meatus and upper nasal meatus, or because the spout is not strong enough to penetrate the crust. At this moment, the patient is better to visit the doctor to receive a computed tomography of the nasal cavity. For considering flushing treatment every day, a single computed tomography is worthy for patient.

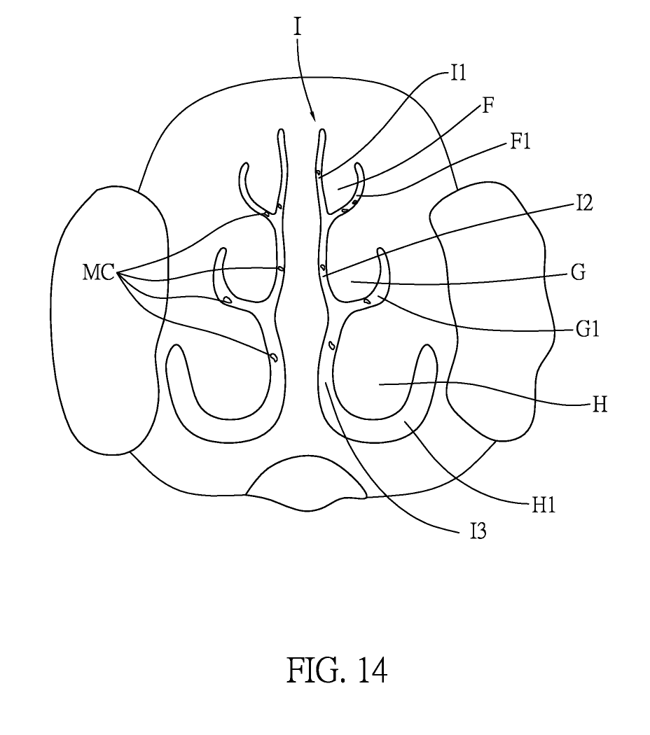

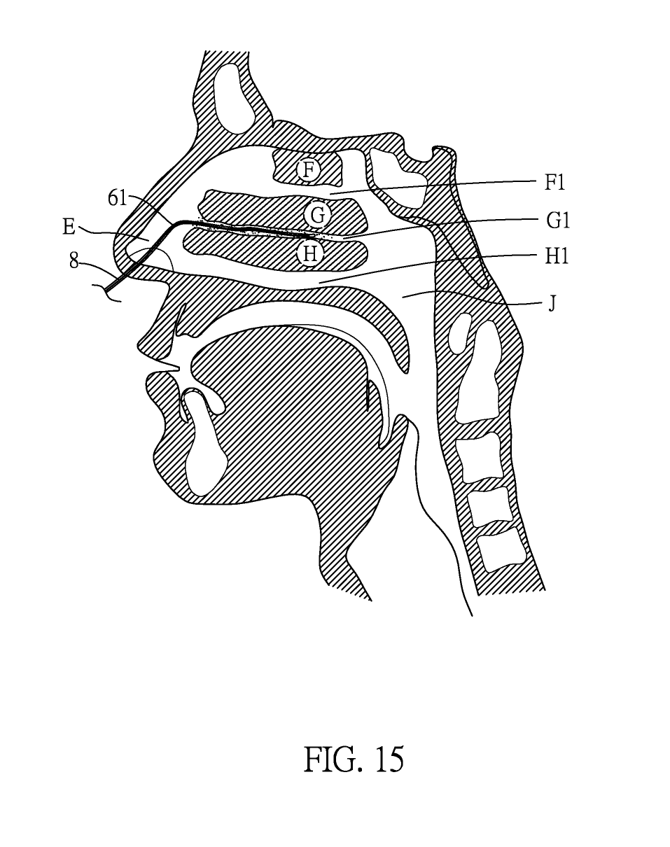

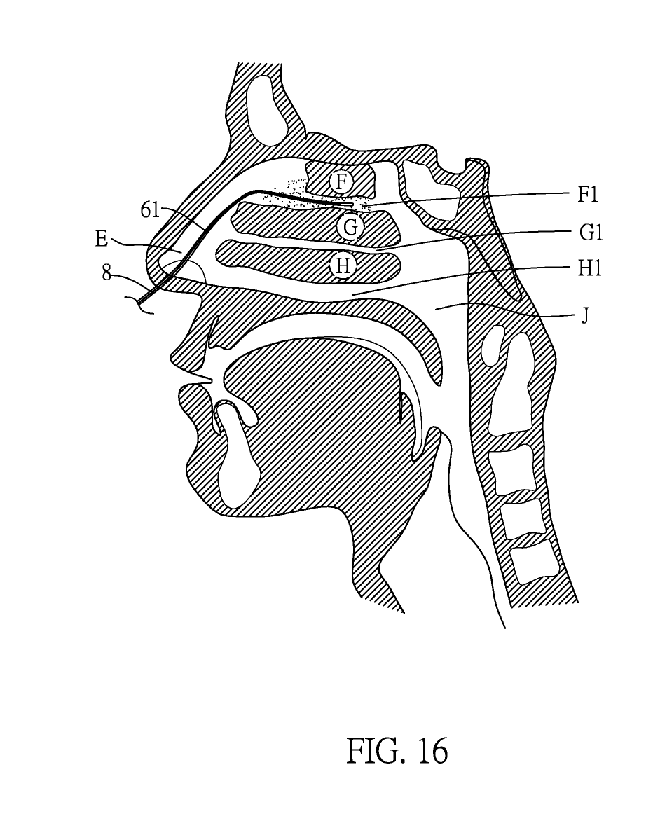

If the result of computed tomography is like FIG. 14, the nasal cavity is divided into three slightly curved slit-like passages by three plate-like structures. The three passages are the upper nasal meatus F1, the middle nasal meatus G1 and the lower nasal meatus H1. The three plate-like structures are the superior turbinate F, the middle turbinate G, and the inferior turbinate H. Although the nasal passage is not completely obstructed and there is no much mucus, the upper turbinate F, the middle turbinate G, and the lower turbinate H appear bigger than normal person, and the upper nasal meatus F1, the middle nasal meatus G1 as well as the lower nasal meatus H1 appear narrower than normal person. The enlarged turbinate should be caused by coating of thick crust resulting in narrowing of nasal meatus. If the outer diameter of the catheter used is bigger than the middle meatus G1, then the unsatisfactory flushing effect may be due to unable to enter the target area. It is necessary to review all the coronal cross-sectional views from front to back, measuring the narrowest point of the insertion route, and select a catheter body 61 smaller than this narrowest point. The design of stylet 8 renders the catheter body 61 a good controllability. As shown in FIG. 14 and FIG. 15, a catheter inserted from the nasal vestibule E to the inferior para-septal space 13, and deviates laterally can reach the middle nasal meatus G1 (FIG. 15) when inserted more than 5-6 cm deep; a catheter inserted from nasal vestibule, through inferior para-septal space 13, middle para-septal space 12 then deviating laterally can reach the superior nasal meatus F1 (FIG. 16) when inserted more than 7-8 cm deep. Because the catheter body 61 has already reached the target area, although the flow rate of the catheter is small and the spouts are weak, it is enough for direct penetration of the crust to achieve a good flushing effect.

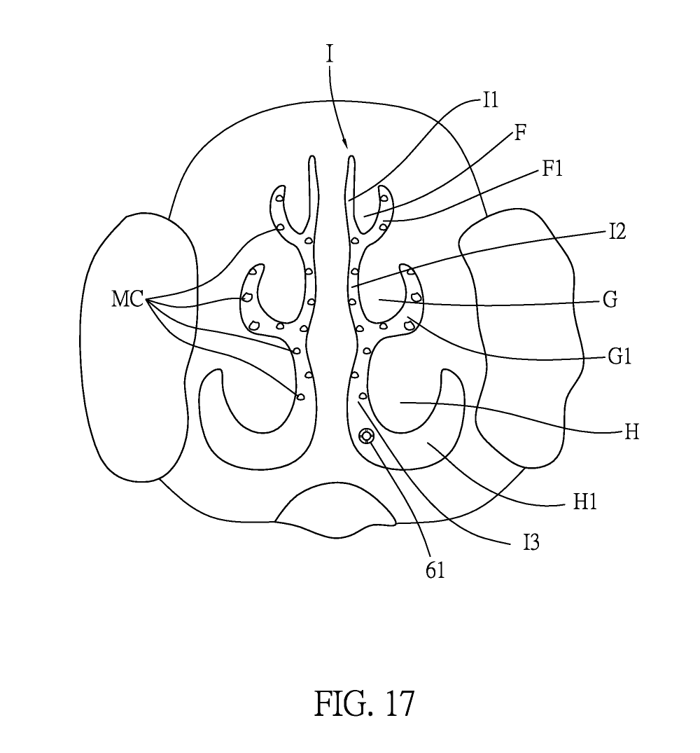

If the result of computed tomography is like FIG. 17, due to the mucus or the solid crust are accumulated from narrow the superior nasal meatus F1 and the middle nasal meatus G1 downward to the inferior para-septal space 13, the respiratory air cannot pass through the upper part of nasal cavity but still can easily pass through the non-obstructed inferior part of nasal cavity. Due to the drying effect of respiratory air, the lower edge of the mucus accumulation becomes a solid hard crust. In such situation, the 30 cm spout of the prior nasal flushing catheter is inadequate for penetrating such hard crust. The patient can choose medium a pressure catheter or high pressure catheter for insertion under through the inferior para-septal space 13 below the solid crust, the medium pressure spouts or high pressure spouts from the catheters may have better penetrating and cleaning effect, and quickly relieve the symptoms of nasal obstruction.

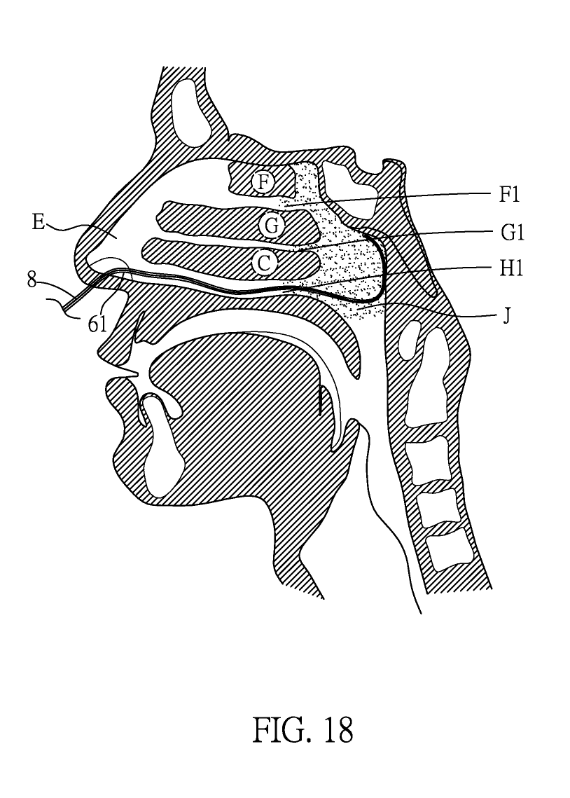

Please refer to FIG. 18, the nasopharynx J is a wide passage for respiratory air, the mucus migrates into the nasopharynx J will quickly transform to solid crust by the drying effect of respiratory air. The single direction spout of the conventional flushing device can only flush the rear wall of the nasopharynx J, but cannot flush the roof and both sides of the nasopharynx J. Moreover, if exerting more pressure, the huge spouts could be accompanied by a large flow to cause choking phenomenon. Even the 30 cm spout of the prior nasal flushing catheter is too weak to penetrating to the thick solid crust. In such situation, the patient can insert a medium pressure or high pressure catheter with a curve tip horizontally through the inferior nasal meatus H1, middle nasal meatus G1 or para-septal space I into the nasopharynx J, then by rotating the catheter to direct the catheter tip up to the roof of nasopharynx. By forceful injection of a syringe 7 to produce stronger spouts, the solid crust in the nasopharynx J can be penetrated resulting in good cleansing effect.

From the above description, a patient can choose a catheter as needed. Due to the presence of the stylet 8, the nasal flushing catheter has better controllability and is easy to enter the superior nasal meatus F1, middle nasal meatus G1, lower nasal meatus H1 and the nasopharynx J to clean mucus and solid crust.

From the above detailed description of the embodiments, it can be known that the nasal flushing catheter of the present invention has the following effects:

1. It is possible to flush the narrow nasal passages directly by a micro-catheter which is easy to be operated.

2. Due to the improvement of the connector 62, a catheter can eject stronger spouts to improving cleansing effect without increasing the diameter.

3. The strength of the spouts can be predicted by the total area of side-holes 613, so that the stronger the spout, the better the effect.

4. Through the controllability of the catheter, the position of the catheter body 61 can be adjusted in the nasopharynx J, thus strong spouts can be ejected to clean the mucus and solid crust within the nasopharynx J.

The nasal flushing catheter of the present invention can overcome the disadvantages of the prior nasal flushing device by better controllability to achieved deep insertion to small deep recess of the nasal cavity and by stronger spouts to penetrate solid crust. This is very important in improving the symptoms of patients with chronic sinusitis. Meanwhile, it is also convenient for the patient in travelling.

The above descriptions are only explanations according to the five preferred embodiments, and should not limit the scope the present invention. It is to be understood that many other possible modifications and variations can be made without departing from the spirit and scope of the invention as hereinafter claimed.

1. A nasal flushing catheter, is adapted to a syringe for flushing the nasal cavity and nasopharynx, wherein
the nasal flushing catheter comprises a catheter body and a connector formed outside of the catheter body;
the catheter body is made of silicone, latex, thermoplastic elastomer or other soft and elastic material, rendering insertion by patient into the nasal cavity and nasopharynx; and
the catheter body has a closed end, an open end on the opposite side, and a plurality of side-holes disposed near the closed end;
wherein the total area of the said plurality of side-holes is not more than 4.123 mm².

2.-9. (canceled)

10. The nasal flushing catheter as claimed in claim 1, wherein the total area of side-holes of the catheter body is between 3.367 mm²˜4.123 mm² in range.

11. The nasal flushing catheter as claimed in claim 1, wherein the total area of side-holes of the catheter body is between 2.381 mm²˜3.367 mm² in range.

12. The nasal flushing catheter as claimed in claim 1, wherein the total area of side-holes of the catheter body is smaller than 2.381 mm².

13.-14. (canceled)
